(19) United States
(12) Patent Application Publication
(10) Pub. No.: US 2023/0242671 A1
(43) Pub. Date: Aug. 3, 2023

(54) MULTISPECIFIC BINDING PROTEINS AND METHODS OF DEVELOPING THE SAME

(71) Applicant: Eli Lilly and Company, Indianapolis, IN (US)

(72) Inventors: Qing CHAI, San Diego, CA (US); Xiufeng WU, Burlingame, CA (US)

(21) Appl. No.: 17/905,520

(22) PCT Filed: Mar. 18, 2021
(86) PCT No.: PCT/US2021/022935

§ 371 Date: Sep. 2, 2022

Related U.S. Application Data
(60) Provisional application No. 62/994,509, filed on Mar. 25, 2020.

(51) Int. Cl. C07K 16/40; C07K 16/28; C07K 16/06

(52) U.S. Cl. CPC C07K 16/40; C07K 16/2845; C07K 16/065; C07K 16/2818; C07K 16/2803; C07K 2317/31; C07K 2317/55; C07K 2317/52; C07K 2317/92; C07K 2317/10

(57) ABSTRACT

Multispecific binding proteins that bind a first antigen and a second antigen and methods of purifying multispecific binding proteins.

The present invention is in the field of medicine, particularly in the field of multispecific binding proteins, such as bispecific antibodies and trispecific binding proteins, used in the treatment of diseases and methods of developing the same.

Multispecific binding proteins are polypeptides which comprise multiple distinct antigen binding domains. Multiple formats of multispecific binding proteins, such as those set forth in WO2001077342, WO2007110205, WO2008024188, WO2009089004, WO2012135345 and WO2016118742, have been disclosed and even tested for the treatment of various autoimmune diseases, cancers, infectious diseases and cardiovascular disease. Although multispecific binding proteins offer the possibility for enhanced therapeutic benefit, for example by targeting multiple antigens, potential of cost savings and improved convenience to patients, their development as therapeutic candidates has been limited.

A factor limiting the advancement of multispecific binding proteins is the complexity of assembling, manufacturing and purifying these molecules. For example, manufacturing of bispecific molecules not only requires the proper assembly of distinct antigen binding domains, but also assembly of the distinct antigen binding domains into a single molecule. Often, during recombinant expression of a multispecific binding protein, a mixture including undesired molecules (e.g., monospecific proteins, single chain pairs, etc.) is expressed. A desired multispecific binding protein must be purified not only from the expression medium, but also from the mixture of undesired molecules. The formation of undesired molecules and need for additional purification steps result in reduced yield of the desired multispecific binding protein and increased overall manufacturing costs.

Attempts at enhancing the development and purification of multispecific binding proteins have been disclosed, for example, as set forth in WO20100151792, WO2013088259 and WO2013136186. However, these disclosures have proven to be limited in addressing the development issues for multispecific binding proteins. For example, in some instances, these disclosures demonstrate one of more of impaired effector function, enhanced immunogenicity concerns, altered assembly, altered affinity and/or reduced pharmacokinetic properties such as half-life. In some instances, the applicability of the disclosure is limited to a specific molecule and/or format. As such, there remains a need for improved multispecific binding proteins and methods of developing the same, which enhances the development of multispecific binding proteins without altering stability or affinity and which is not attendant upon unacceptable immunogenicity.

Accordingly, the present disclosure addresses one or more of the above needs by providing improved multispecific binding proteins and methods of developing the same. Embodiments of the multispecific binding proteins and methods of the present disclosure provide for enhanced purification of the desired multispecific binding protein, preserve and/or enhance assembly of the molecule, decreased protein aggregation, improved physical stability and are not attendant upon increased immunogenicity risk, altered effector function and/or altered pharmacokinetic properties. Additionally, embodiments of the present disclosure preserve affinity of the multispecific binding protein and reduces or eliminates undesired binding of kappa light chain to purification reagent. Furthermore, embodiments of the present disclosure do not add time and/or cost to the purification process or development process as a whole.

Accordingly, in particular embodiments, the present disclosure provides a multispecific binding protein that binds a first antigen and a second antigen. According to some embodiments, multispecific binding proteins are provided that bind a first antigen and a second antigen, the multispecific binding protein comprising a first antigen binding domain comprising a first light chain Fab region and a first heavy chain Fab region, wherein the first light chain Fab region is a kappa light chain and comprises: a lysine at amino acid residue 143 (EU numbering) and a lysine at amino acid residue 199 (EU numbering); a lysine at amino acid residue 143 (EU numbering), a lysine at amino acid residue 199 (EU numbering), and an alanine at amino acid residue 109 (EU numbering); a lysine at amino acid residue 143 (EU numbering), a lysine at amino acid residue 199 (EU numbering), and an aspartic acid at amino acid residue 110 (EU numbering); a lysine at amino acid residue 143 (EU numbering), a lysine at amino acid residue 199 (EU numbering), an alanine at amino acid residue 109 (EU numbering), and an aspartic acid at amino acid residue 110 (EU numbering); an aspartic acid at amino acid residue 110 (EU numbering) and a lysine at amino acid residue 143 (EU numbering); an aspartic acid at amino acid residue 110 (EU numbering), a lysine at amino acid residue 143 (EU numbering) and an alanine at amino acid residue 109 (EU numbering); an aspartic acid at amino acid residue 110 (EU numbering) and a lysine at amino acid residue 199 (EU numbering); an aspartic acid at amino acid residue 110 (EU numbering), a lysine at amino acid residue 199 (EU numbering) and an alanine at amino acid residue 109 (EU numbering); an alanine at amino acid residue 109 (EU numbering) and a lysine at amino acid residue 143 (EU numbering); an alanine at amino acid residue 109 (EU numbering) and a lysine at amino acid residue 199 (EU numbering); or an alanine at amino acid residue 109 (EU numbering) and an aspartic acid at amino acid residue 110 (EU numbering); and a second antigen binding domain comprising a second light chain Fab region and a second heavy chain Fab region, wherein the first antigen binding domain binds the first antigen and the second antigen binding domain binds the second antigen. According to some such embodiments, if the first light chain Fab region comprises a lysine at amino acid residue 143 (EU numbering) and a lysine at amino acid residue 199 (EU numbering), then the second light chain Fab region does not comprise a lysine at amino acid residue 143 (EU numbering) and a lysine at amino acid residue 199 (EU numbering); if the first light chain Fab region comprises a lysine at amino acid residue 143 (EU numbering), a lysine at amino acid residue 199 (EU numbering), and an alanine at amino acid residue 109 (EU numbering), then the second light chain Fab region does not comprise a lysine at amino acid residue 143 (EU numbering), a lysine at amino acid residue 199 (EU numbering), and an alanine at amino acid residue 109 (EU numbering); if the first light chain Fab region comprises a lysine at amino acid residue 143 (EU numbering), a lysine at amino acid residue 199 (EU numbering), and an aspartic acid at amino acid residue 110 (EU numbering), then the second light chain Fab region does not comprise a lysine at amino acid residue 143 (EU numbering), a lysine at amino acid residue 199 (EU numbering), and an aspartic acid at amino acid residue 110 (EU numbering); if the first light chain Fab region comprises a lysine at amino acid residue 143 (EU numbering), a lysine at amino acid residue 199 (EU numbering), an alanine at amino acid residue 109 (EU numbering), and an aspartic acid at amino acid residue 110 (EU numbering), then the second light chain Fab region does not comprise a lysine at amino acid residue 143 (EU numbering), a lysine at amino acid residue 199 (EU numbering), an alanine at amino acid residue 109 (EU numbering); if the first light chain Fab region comprises an aspartic acid at amino acid residue 110 (EU numbering) and a lysine at amino acid residue 143 (EU numbering), then the second light chain Fab region does not comprise an aspartic acid at amino acid residue 110 (EU numbering) and a lysine at amino acid residue 143 (EU numbering); if the first light chain Fab region comprises an aspartic acid at amino acid residue 110 (EU numbering), a lysine at amino acid residue 143 (EU numbering) and an alanine at amino acid residue 109 (EU numbering), then the second light chain Fab region does not comprise an aspartic acid at amino acid residue 110 (EU numbering), a lysine at amino acid residue 143 (EU numbering) and an alanine at amino acid residue 109 (EU numbering); if the first light chain Fab region comprises an aspartic acid at amino acid residue 110 (EU numbering) and a lysine at amino acid residue 199 (EU numbering), then the second light chain Fab region does not comprise an aspartic acid at amino acid residue 110 (EU numbering) and a lysine at amino acid residue 199 (EU numbering); if the first light chain Fab region comprises an aspartic acid at amino acid residue 110 (EU numbering), a lysine at amino acid residue 199 (EU numbering) and an alanine at amino acid residue 109 (EU numbering), then the second light chain Fab region does not comprise an aspartic acid at amino acid residue 110 (EU numbering), a lysine at amino acid residue 199 (EU numbering) and an alanine at amino acid residue 109 (EU numbering); if the first light chain Fab region comprises an alanine at amino acid residue 109 (EU numbering) and a lysine at amino acid residue 143 (EU numbering), then the second light chain Fab region does not comprise an alanine at amino acid residue 109 (EU numbering) and a lysine at amino acid residue 143 (EU numbering); if the first light chain Fab region comprises an alanine at amino acid residue 109 (EU numbering) and a lysine at amino acid residue 199 (EU numbering), then the second light chain Fab region does not comprise an alanine at amino acid residue 109 (EU numbering) and a lysine at amino acid residue 199 (EU numbering); and if the first light chain Fab region comprises an alanine at amino acid residue 109 (EU numbering) and an aspartic acid at amino acid residue 110 (EU numbering), then the second light chain Fab region does not comprise an alanine at amino acid residue 109 (EU numbering) and an aspartic acid at amino acid residue 110 (EU numbering).

According to some embodiments, the multispecific binding protein comprises a first antigen binding domain comprising a first light chain Fab region and a first heavy chain Fab region, wherein the first light chain Fab region is a kappa light chain and comprises a lysine at amino acid residue 143 (EU numbering) and a lysine at amino acid residue 199 (EU numbering); and a second antigen binding domain comprising a second light chain Fab region and a second heavy chain Fab region, wherein the first antigen binding domain binds the first antigen and the second antigen binding domain binds the second antigen. In some embodiments, the first light chain Fab region further comprises an aspartic acid at amino acid residue 110 (EU numbering). In some embodiments, the first light chain Fab region further comprises an alanine at amino acid residue 109 (EU numbering).

According to further embodiments of the multispecific binding proteins provided herein, the multispecific binding protein comprises a first antigen binding domain comprising a first light chain Fab region and a first heavy chain Fab region, wherein the first light chain Fab region is a kappa light chain and comprises an aspartic acid at amino acid residue 110 (EU numbering) and a lysine at amino acid residue 143 (EU numbering); and a second antigen binding domain comprising a second light chain Fab region and a second heavy chain Fab region, wherein the first antigen binding domain binds the first antigen and the second antigen binding domain binds the second antigen. In some embodiments, the first light chain Fab region further comprises an alanine at amino acid residue 109 (EU numbering).

According to embodiments of the multispecific binding proteins provided herein, the multispecific binding protein comprises a first antigen binding domain comprising a first light chain Fab region and a first heavy chain Fab region, wherein the first light chain Fab region is a kappa light chain and comprises an aspartic acid at amino acid residue 110 (EU numbering) and a lysine at amino acid residue 199 (EU numbering); and a second antigen binding domain comprising a second light chain Fab region and a second heavy chain Fab region, wherein the first antigen binding domain binds the first antigen and the second antigen binding domain binds the second antigen. In some embodiments, the first light chain Fab region further comprises an alanine at amino acid residue 109 (EU numbering).

According to embodiments of the multispecific binding proteins provided herein, the multispecific binding protein comprises a first antigen binding domain comprising a first light chain Fab region and a first heavy chain Fab region, wherein the first light chain Fab region is a kappa light chain and comprises an alanine at amino acid residue 109 (EU numbering) and a lysine at amino acid residue 143 (EU numbering); and a second antigen binding domain comprising a second light chain Fab region and a second heavy chain Fab region, wherein the first antigen binding domain binds the first antigen and the second antigen binding domain binds the second antigen.

According to some embodiments of the multispecific binding proteins provided herein, the multispecific binding protein comprises a first antigen binding domain comprising a first light chain Fab region and a first heavy chain Fab region, wherein the first light chain Fab region is a kappa light chain and comprises an alanine at amino acid residue 109 (EU numbering) and a lysine at amino acid residue 199 (EU numbering); and a second antigen binding domain comprising a second light chain Fab region and a second heavy chain Fab region, wherein the first antigen binding domain binds the first antigen and the second antigen binding domain binds the second antigen.

According to even further embodiments of the multispecific binding proteins provided herein, the multispecific binding protein comprises a first antigen binding domain comprising a first light chain Fab region and a first heavy chain Fab region, wherein the first light chain Fab region is a kappa light chain and comprises an alanine at amino acid residue 109 (EU numbering) and an aspartic acid at amino acid residue 110 (EU numbering); and a second antigen binding domain comprising a second light chain Fab region and a second heavy chain Fab region, wherein the first antigen binding domain binds the first antigen and the second antigen binding domain binds the second antigen.

According to some embodiments of the multispecific binding proteins of the present disclosure, the first antigen binding domain of the multispecific binding protein further comprises a first heavy chain Fc region. In even further embodiments of the multispecific binding proteins of the present disclosure, the first heavy chain Fc region comprises a human IgG1, a human IgG2 or a human IgG4 constant region. In some embodiments of the multispecific binding proteins of the present disclosure, the second antigen binding domain further comprises a second heavy chain Fc region. In even further embodiments of the multispecific binding proteins of the present disclosure, the second heavy chain Fc region comprises a human IgG1, a human IgG2 or a human IgG4 constant region. In some embodiments of the multispecific binding proteins of the present disclosure, the first heavy chain Fc region comprises an arginine at amino acid residue 311 (EU numbering) and a glutamic acid at amino acid residue 317 (EU numbering). According to some embodiments of the multispecific binding proteins of the present disclosure the second heavy chain Fc region comprises an arginine at amino acid residue 311 (EU numbering) and a glutamic acid at amino acid residue 317 (EU numbering). In some embodiments of the multispecific binding proteins of the present disclosure both the first and second heavy chain Fc regions comprise a human IgG1 constant region; both comprise a human IgG2 constant region; or both comprise a human IgG4 constant region. In even further embodiments of the multispecific binding proteins of the present disclosure both the first and second heavy chain Fc regions comprise an arginine at amino acid residue 311 (EU numbering) and a glutamic acid at amino acid residue 317 (EU numbering). According to some embodiments of the multispecific binding proteins of the present disclosure the second light chain Fab region does not comprise an alanine at amino acid residue 109; does not comprise an aspartic acid at amino acid residue 110; does not comprise a lysine at amino acid residue 143; or does not comprise a lysine at amino acid residue 199. In some embodiments of the multispecific binding proteins of the present disclosure the second light chain Fab region does not comprise an alanine at amino acid residue 109; does not comprise an aspartic acid at amino acid residue 110; does not comprise a lysine at amino acid residue 143; and does not comprise a lysine at amino acid residue 199. According to some embodiments of the multispecific binding proteins of the present disclosure the second light chain Fab region is a Kappa light chain. Further, according to some embodiments of the multispecific binding proteins of the present disclosure the second light chain Fab region is a Lambda light chain.

According to some embodiments, the multispecific binding protein comprises a bispecific binding protein. According to some such embodiments, the bispecific binding protein is an immunoglobulin heteromab. In some more specific embodiments, the immunoglobulin heteromab is an IgG heteromab. According to even further embodiments, the multispecific binding protein comprises a multispecific binding protein.

Furthermore, embodiments of the present disclosure also provide pharmaceutical compositions comprising a multispecific binding protein of the present disclosure and one or more pharmaceutically acceptable carriers, diluents or excipients.

According to additional embodiments of the present disclosure, methods of purifying multispecific binding proteins of the present disclosure is provided. According to some such embodiments, the method comprises introducing into the first antigen binding domain a first light chain Fab region comprising a lysine at amino acid residue 143 (EU numbering) and a lysine at amino acid residue 199 (EU numbering), wherein the first light chain Fab region is a kappa light chain; expressing the multispecific binding protein, wherein the first antigen binding domain assembles with the second antigen binding domain; subjecting the multispecific binding protein to an affinity chromatography column; and recovering purified multispecific binding protein. According to some such embodiments, the step of introducing further comprises introducing into the first antigen binding domain an alanine at amino acid residue 109 (EU numbering). In some embodiments, the step of introducing further comprises introducing into the first antigen binding domain an aspartic acid at amino acid residue 110 (EU numbering).

Additional embodiments of methods of purifying multispecific binding proteins of the present disclosure are provided which comprise introducing into the first antigen binding domain a first light chain Fab region comprising an aspartic acid at amino acid residue 110 (EU numbering) and a lysine at amino acid residue 143 (EU numbering), wherein the first light chain Fab region is a kappa light chain; expressing the multispecific binding protein, wherein the first antigen binding domain assembles with the second antigen binding domain; subjecting the multispecific binding protein to an affinity chromatography column; and recovering purified multispecific binding protein. According to some embodiments, the step of introducing further comprises introducing into the first antigen binding domain an alanine at amino acid residue 109 (EU numbering).

Additional embodiments of methods of purifying multispecific binding proteins of the present disclosure are provided which comprise introducing into the first antigen binding domain a first light chain Fab region comprising an aspartic acid at amino acid residue 110 (EU numbering) and a lysine at amino acid residue 199 (EU numbering), wherein the first light chain Fab region is a kappa light chain; expressing the multispecific binding protein, wherein the first antigen binding domain assembles with the second antigen binding domain; subjecting the multispecific binding protein to an affinity chromatography column; and recovering purified multispecific binding protein. According to some embodiments, the step of introducing further comprises introducing into the first antigen binding domain an alanine at amino acid residue 109 (EU numbering).

According to additional embodiments, methods of purifying multispecific binding proteins of the present disclosure are provided comprising introducing into the first antigen binding domain a first light chain Fab region comprising an alanine at amino acid residue 109 (EU numbering) and a lysine at amino acid residue 143 (EU numbering), wherein the first light chain Fab region is a kappa light chain; expressing the multispecific binding protein, wherein the first antigen binding domain assembles with the second antigen binding domain; subjecting the multispecific binding protein to an affinity chromatography column; and recovering purified multispecific binding protein.

Additional embodiments of methods of purifying multispecific binding proteins of the present disclosure are provided comprising introducing into the first antigen binding domain a first light chain Fab region comprising an alanine at amino acid residue 109 (EU numbering) and a lysine at amino acid residue 199 (EU numbering), wherein the first light chain Fab region is a kappa light chain; expressing the multispecific binding protein, wherein the first antigen binding domain assembles with the second antigen binding domain; subjecting the multispecific binding protein to an affinity chromatography column; and recovering purified multispecific binding protein.

Even further embodiments of methods of purifying multispecific binding proteins of the present disclosure are provided comprising introducing into the first antigen binding domain a first light chain Fab region comprising an alanine at amino acid residue 109 (EU numbering) and an aspartic acid at amino acid residue 110 (EU numbering), wherein the first light chain Fab region is a kappa light chain; expressing the multispecific binding protein, wherein the first antigen binding domain assembles with the second antigen binding domain; subjecting the multispecific binding protein to an affinity chromatography column; and recovering purified multispecific binding protein.

According to some embodiments of the methods of purifying multispecific binding proteins of the present disclosure the step of introducing further comprises introducing into the first antigen binding domain a first heavy chain Fc region. In even further embodiments of the methods of the present disclosure, the first heavy chain Fc region comprises a human IgG1, a human IgG2 or a human IgG4 constant region. In some embodiments of the methods of the present disclosure, the step of introducing further comprises introducing into the second antigen binding domain a second heavy chain Fc region. In even further embodiments of the methods of the present disclosure, the second heavy chain Fc region comprises a human IgG1, a human IgG2 or a human IgG4 constant region. According to some embodiments of the methods of the present disclosure, the step of introducing further comprises introducing into the first heavy chain Fc region an arginine at amino acid residue 311 (EU numbering) and a glutamic acid at amino acid residue 317 (EU numbering). In some embodiments of the methods of the present disclosure, the step of introducing further comprises introducing into the second heavy chain Fc region an arginine at amino acid residue 311 (EU numbering) and a glutamic acid at amino acid residue 317 (EU numbering). According to some embodiments of the methods of the present disclosure, both the first heavy chain Fc region and the second heavy chain Fc region comprise a human IgG1 constant region; both comprise a human IgG2 constant region; or both comprise a human IgG4 constant region. In some embodiments of the methods of the present disclosure, the step of introducing further comprises introducing, into both the first heavy chain Fc region and the second heavy chain Fc region, arginine at amino acid residues 311 (EU numbering) and glutamic acid at amino acid residues 317 (EU numbering). In some embodiments of the methods of the present disclosure, the second light chain Fab region does not comprise an alanine at amino acid residue 109; does not comprise an aspartic acid at amino acid residue 110; does not comprise a lysine at amino acid residue 143; or does not comprise a lysine at amino acid residue 199. In some embodiments of the methods of the present disclosure, the second light chain Fab region does not comprise an alanine at amino acid residue 109; does not comprise an aspartic acid at amino acid residue 110; does not comprise a lysine at amino acid residue 143; and does not comprise a lysine at amino acid residue 199. In some embodiments of the methods of the present disclosure, the second light chain Fab region is a Kappa light chain. In further embodiments of the methods of the present disclosure, the second light chain Fab region is a Lambda light chain.

According to further embodiments of the methods of the present disclosure, the affinity chromatography column comprises a kappa affinity ligand. In some embodiments of the methods of the present disclosure the affinity chromatography column comprises a lambda affinity ligand. According to some embodiments of the methods of the present disclosure, the affinity chromatography column comprises Protein A. In some embodiments of the methods of the present disclosure, the second light chain Fab region binds to the affinity chromatography column with greater affinity than the first light chain Fab region. In even further embodiments of the methods of the present disclosure, the first light chain Fab region does not bind to the affinity chromatography column.

According to even further embodiments of the methods of purifying multispecific binding proteins of the present disclosure, the methods further comprise subjecting the purified multispecific binding protein to a second affinity chromatography column after the step of recovering purified multispecific binding protein; and recovering purified multispecific binding protein after the step of subjecting the purified multispecific binding protein to a second affinity chromatography column. According to some embodiments, the second affinity chromatography column comprises a kappa affinity ligand. In some embodiments, the second affinity chromatography column comprises a lambda affinity ligand. According to some embodiments, the second affinity chromatography column comprises Protein A. In even some further embodiments, the second light chain Fab region binds to the second affinity chromatography column with greater affinity than the first light chain Fab region. Even further, in some embodiments, the first light chain Fab region does not bind to the second affinity chromatography column.

According to even further embodiments, the present disclosure provides a method of making a multispecific binding protein of the present disclosure. In some such embodiments, such methods comprise a multispecific binding protein of the present invention prepared according to a process, wherein said process comprises cultivating a host cell comprising a polynucleotide sequence, the polynucleotide sequence encoding a first antigen binding domain and a second antigen binding domain of the present disclosure, under conditions such that the multispecific binding protein is expressed, and recovering from said host cell a multispecific binding protein of the present invention. According to some embodiments, the polynucleotide sequence comprises a single vector encoding the first antigen binding domain and the second antigen binding domain. According to further embodiments, the polynucleotide sequence comprises a first vector encoding the first antigen binding domain and a second vector comprising the second antigen binding domain. In some embodiments, the method of the present disclosure further comprises the steps of subjecting the recovered multispecific binding protein to an affinity chromatography column and recovering purified multispecific binding protein. In some embodiments, the affinity chromatography column comprises Protein A. In some embodiments, the affinity chromatography column comprises a kappa affinity ligand. In some embodiments, the affinity chromatography column comprises a lambda affinity ligand. According to some embodiments, the first antigen binding domain comprises a first light chain Fab region and the second antigen binding domain comprises a second light chain Fab region, the second light chain Fab region binding to the affinity chromatography column with greater affinity than the first light chain Fab region. In some embodiments, the first light chain Fab region does not bind to the affinity chromatography column. According to even further embodiments, the method of the present disclosure further comprises the steps of subjecting the purified multispecific binding protein to a second affinity chromatography column after the step of recovering purified multispecific binding protein and recovering purified multispecific binding protein after the step of subjecting the purified multispecific binding protein to a second affinity chromatography column. In some embodiments, the second affinity chromatography column comprises Protein A. In some embodiments, the second affinity chromatography column comprises a kappa affinity ligand. In some embodiments, the second affinity chromatography column comprises a lambda affinity ligand. According to some embodiments, the first antigen binding domain comprises a first light chain Fab region and the second antigen binding domain comprises a second light chain Fab region, the second light chain Fab region binding to the second affinity chromatography column with greater affinity than the first light chain Fab region. In some embodiments, the first light chain Fab region does not bind to the second affinity chromatography column

In even further embodiments, the present disclosure provides multispecific binding proteins for use in therapy. In some embodiments, the present disclosure provides multispecific binding proteins for use in the treatment of a medical condition. In some such embodiments, the medical condition is one of cancer, cardiovascular disease, autoimmune disease or a neurodegenerative disease.

In further embodiments, the present disclosure provides multispecific binding proteins for use in the manufacture of a medicament. In some embodiments, the present disclosure provides multispecific binding proteins for use in the manufacture of a medicament for therapy. In further embodiments, the present disclosure provides multispecific binding proteins for use in the manufacture of a medicament for the treatment of a medical condition. In some such embodiments, the medical condition is one of cancer, cardiovascular disease, autoimmune disease or a neurodegenerative disease.

The term “multispecific binding protein”, as used herein, refers to a molecule having two or more distinct antigen-binding domains. Multispecific binding proteins of the present disclosure bind two or more different antigens or two or more different epitopes of the same antigen. Embodiments of multispecific binding proteins of the present disclosure include bispecific antibodies, as well as trispecific or tetraspecific binding molecules as known in the field as well as single chain multispecific binding molecules including diabodies. Multispecific binding proteins of the instant disclosure can differ in size and geometry and can comprise multiple formats as known in the art.

As referred to herein, “antigen binding domain” refers to a portion of a multispecific binding protein that comprises amino acid residues that interact with, and confer specificity for, the respective antigen. Antigen binding domains of multispecific binding proteins of the present disclosure include a light chain Fab region and a heavy chain Fab region. Both the heavy and light chain Fab regions include a variable portion, at the amino-terminus, comprising CDRs interspersed with regions that are more conserved termed framework regions. Both the heavy and light chain Fab regions also include a conserved region (e.g., a C_{L} for the light chain and CH₁ for the heavy chain Fab region, as known in the field). The light chain Fab regions are classified as kappa or lambda, as known in the art.

Some embodiments of multispecific binding proteins of the present disclosure include heavy chain Fc regions linked at the carboxy terminus of the heavy chain Fab region (e.g., forming a heavy chain as known in the field). Heavy chain Fc regions of the present disclosure are classified as gamma and define the isotype of heavy chain as IgG and one of subclasses IgG1, IgG2, IgG3 or IgG4. The heavy chain Fc region may further comport an effector function (as known in the field) upon the multispecific binding protein.

According to some particular embodiments, multispecific binding proteins of the instant disclosure comprise an IgG heteromab molecule, or fragment thereof. As known in the art, IgG heteromab molecules comprise archetypical Fab architecture and IgG structure (with one Fab “arm”, or antigen binding domain, binding the first antigen and the other Fab “arm”, or antigen binding domain, binding the second antigen).

The term “EU numbering”, which is recognized in the art, refers to a system of numbering amino acid residues of immunoglobulin molecules. EU numbering is described, for example, at Kabat et al., Sequences of Proteins of Immunological Interest, 5th Ed. Public Health Service, National Institutes of Health, Bethesda, Md. (1991); Edelman, G. M, et al., Proc. Natl. Acad. USA, 63, 78-85 (1969); and http://www.imgt.org/IMGTScientificChart/Numbering/Hu_IGHGnber.html #refs. The term “Kabat numbering” is recognized in the art as referring to a system of numbering amino acid residues which are more variable (i.e., hypervariable) than other amino acid residues in heavy and light chain variable regions (see, for example, Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)). The term “North numbering”, refers to a system of numbering amino acid residues which are more variable (i.e., hypervariable) than other amino acid residues in heavy and light chain variable regions and is based, at least in part, on affinity propagation clustering with a large number of crystal structures, as described in (North et al., A New Clustering of Antibody CDR Loop Conformations, Journal of Molecular Biology, 406:228-256 (2011).

As used herein, the term “affinity chromatography” refers to a chromatographic method for separating biochemical mixtures (e.g., a multispecific binding protein and undesired biomolecule species) based on specific, reversible interactions between biomolecules. Exemplary embodiments of affinity chromatography include Protein A affinity columns, kappa affinity ligand chromatograph (such as CaptureSelect™ KappaXL™, KappaSelect™, KappaXP™) or lambda affinity ligand chromatography.

A “parent” or “parental” molecule as referred to herein, is a molecule encoded by an amino acid sequence which is used in the preparation of one of the exemplified embodiments set forth herein, for example through amino acid substitutions and structural alteration. A parental molecule may comprise, for example, a murine antibody, or fragment thereof, or a binding protein derived through phage display or transgenic non-human animals, for example.

A multispecific binding protein of the present disclosure can be incorporated into a pharmaceutical composition which can be prepared by methods well known in the art and which comprise a multispecific binding protein of the present disclosure and one or more pharmaceutically acceptable carrier(s) and/or diluent(s) (e.g., Remington, The Science and Practice of Pharmacy, 22^{nd} Edition, Loyd V., Ed., Pharmaceutical Press, 2012, which provides a compendium of formulation techniques as are generally known to practitioners). Suitable carriers for pharmaceutical compositions include any material which, when combined with the multispecific binding protein, retains the molecule's activity and is non-reactive with the patient's immune system.

Expression vectors capable of directing expression of genes to which they are operably linked are well known in the art. Expression vectors can encode a signal peptide that facilitates secretion of the polypeptide(s) from a host cell. The signal peptide can be an immunoglobulin signal peptide or a heterologous signal peptide. Each of the expressed polypeptides may be expressed independently from different promoters to which they are operably linked in one vector or, alternatively, may be expressed independently from different promoters to which they are operably linked in multiple vectors. The expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences.

A host cell refers to cells stably or transiently transfected, transformed, transduced or infected with one or more expression vectors expressing one or more polypeptide chain of a multispecific binding protein of the present disclosure. Creation and isolation of host cell lines producing binding proteins of the present disclosure can be accomplished using standard techniques known in the art. Mammalian cells are preferred host cells for expression of multispecific binding proteins of the present disclosure. Particular mammalian cells include HEK 293, NSO, DG-44, and CHO. Preferably, the binding proteins are secreted into the medium in which the host cells are cultured, from which the binding proteins can be recovered or purified by for example using conventional techniques. For example, the medium may be applied to and eluted from a Protein A affinity chromatography column and/or a kappa affinity ligand or lambda affinity ligand chromatography column. Undesired biomolecule species including soluble aggregate and multimers may be effectively removed by common techniques, including size exclusion, hydrophobic interaction, ion exchange, or hydroxyapatite chromatography. The product may be immediately frozen, for example at −70° C., refrigerated, or may be lyophilized. Various methods of protein purification may be employed and such methods are known in the art and described, for example, in Deutscher, Methods in Enzymology 182: 83-89 (1990) and Scopes, Protein Purification: Principles and Practice, 3rd Edition, Springer, N.Y. (1994).

### EXAMPLES

### Expression and Purification of Exemplified Multispecific Binding Proteins

An exemplified multispecific binding protein of the present disclosure, comprising an IgG heteromab format having a first antigen binding domain binding cMet and a second antigen binding domain binding BHA10, may be expressed and purified essentially as follows. Briefly, first light and heavy chain Fab regions are cloned in expression vectors, such as pEHG1 and pEHK expression vectors, containing human G1 allotype constant region and the human kappa light chain constant region, respectively. Both vectors house the murine kappa leader sequences to drive secretion (WO2014/150973 A1; Lewis S. M., et al., 2014 Nat. Biotechnol. 32, 191-8).

Amino acid residue changes may be introduced into the binding domains via methods known in the art including: lightchain, Quickchange site-directed mutagenesis kit (Stratagene, La Jolla, Calif.), codon-optimized coding regions synthesized de novo (into a single or separate vectors), and the like. The EU-numbering convention may be used to determine the mutation location.

Exemplified modified kappa light chain Fab region and heavy chain Fc region formats of the present disclosure are provided in Tables 1a and 1b, respectively (amino acid modifications are numbered based on EU numbering).

TABLE 1a
Exemplified Modified Kappa Light Chain Fab Region Formats

| | |
|---|---|
| V110D + E143K + Q199K | DKK |
| T109A + V110D + Q199K | ADK |
| V110D + Q199K | DK |
| E143K + Q199K | KK |

TABLE 1b
Exemplified Modified Heavy Chain Fc Region Formats

| | |
|---|---|
| Q311R + K317E | RE |

Embodiments of various combinations of IgG heteromabs, comprising heavy and light chain formats of Tables 1a and 1b, are provided in Table 2. Exemplified IgG heteromabs include a first antigen binding domain binding cMet and a second antigen binding domain binding BHA10; or a first antigen binding domain binding PD-1 and a second antigen binding domain binding Tigit. Exemplified IgG heteromabs of Table 2 include different combinations of the modified heavy and light chain formats (of Tables 1a and 1b) comprising the first and second antigen binding domains, respectively, to assess the impact, if any, on expression, assembly and purification based on orientation of formats. Parental monoclonal antibodies) and IgG1 heteromab molecules (e.g., molecules not including a modified light or heavy chain format as set forth in Tables 1a and 1b) are also assessed as controls.

An appropriate host cell, such as CHO, is transiently transfected with an expression system for secreting the exemplified IgG heteromabs of Table 2. The exemplified IgG heteromab is detected in clarified medium, into which the exemplified IgG heteromabs are secreted, by absorbance at 280 nm. As demonstrated in Table 2, the expression levels of the modified kappa light chain Fab antibody formats and the modified heavy chain Fc antibody formats of Table 1 are comparable to the respective parental antibodies. Thus, modification according to the formats of Tables 1a and 1b did not negatively impact expression levels, and in some instances improved expression titers.

TABLE 2
Quantification of expression levels of multispecific
binding molecules comprising modified kappa light
Fab and heavy chain Fc region formats

| Format | Titer (mg/L) |
|---|---|
| cMet parental mAb | 349.26 |
| (HC amino acid sequence of SEQ ID NO: 1; | |
| LC amino acid sequence of SEQ ID NO: 2) | |
| cMet-BAH10 parental IgG1 heteromab | 96.63 |
| (cMet HC amino acid sequence of SEQ ID NO: 1 and | |
| LC amino acid sequence of SEQ ID NO: 2; | |
| BAH10 HC amino acid sequence of SEQ ID NO: 3 and | |
| LC amino acid sequence of SEQ ID NO: 4) | |
| cMet mAb (RE on both HCs) | 376.32 |
| (HC amino acid sequence of SEQ ID NO: 5; | |
| LC amino acid sequence of SEQ ID NO: 2) | |
| cMet mAb (DKK on both LCs) | 141.5 |
| (HC amino acid sequence of SEQ ID NO: 6; | |
| LC amino acid sequence of SEQ ID NO: 7) | |
| cMet-BAH10 IgG1 heteromab | 96.66 |
| (RE on cMet heavy chain Fc region only) | |
| (cMet HC amino acid sequence of SEQ ID NO: 8 and | |
| LC amino acid sequence of SEQ ID NO: 2; | |
| BAH10 HC amino acid sequence of SEQ ID NO: 3 and | |
| LC amino acid sequence of SEQ ID NO: 4) | |
| cMet-BAH10 IgG1 heteromab | 177.51 |
| (DKK on cMet light chain Fab region only) | |
| (cMet HC amino acid sequence of SEQ ID NO: 1 and | |
| LC amino acid sequence of SEQ ID NO: 7; | |
| BAH10 HC amino acid sequence of SEQ ID NO: 3 and | |
| LC amino acid sequence of SEQ ID NO: 4) | |
| cMet-BAH10 IgG1 heteromab | 171.33 |
| ADK on cMet light chain Fab region only) | |
| (cMet HC amino acid sequence of SEQ ID NO: 1 and | |
| LC amino acid sequence of SEQ ID NO: 9; | |
| BAH10 HC amino acid sequence of SEQ ID NO: 3 and | |
| LC amino acid sequence of SEQ ID NO: 4) | |
| PD-1-TIGIT parental IgG1 heteromab | 107.01 |
| (PD-1 HC amino acid sequence of SEQ ID NO: 10 and | |
| LC amino acid sequence of SEQ ID NO: 11; | |
| TIGIT HC amino acid sequence of SEQ ID NO: 12 and | |
| LC amino acid sequence of SEQ ID NO: 13) | |
| PD-1-TIGIT IgG1 heteromab | 99.42 |
| (DKK on PD-1 light chain Fab region only) | |
| (PD-1 HC amino acid sequence of SEQ ID NO: 10 and | |
| LC amino acid sequence of SEQ ID NO: 14; | |
| TIGIT HC amino acid sequence of SEQ ID NO: 12 and | |
| LC amino acid sequence of SEQ ID NO: 13) | |
| PD-1-TIGIT IgG1 heteromab | 80.7 |
| (ADK on PD-1 light chain Fab region only) | |
| (PD-1 HC amino acid sequence of SEQ ID NO: 10 and | |
| LC amino acid sequence of SEQ ID NO: 15; | |
| TIGIT HC amino acid sequence of SEQ ID NO: 12 and | |
| LC amino acid sequence of SEQ ID NO: 13) | |
| PD-1-TIGIT IgG1 heteromab | 108.81 |
| (DK on PD-1 light chain Fab region only) | |
| (PD-1 HC amino acid sequence of SEQ ID NO: 10 and | |
| LC amino acid sequence of SEQ ID NO: 16; | |
| TIGIT HC amino acid sequence of SEQ ID NO: 12 and | |
| LC amino acid sequence of SEQ ID NO: 13) | |
| PD-1-TIGIT IgG1 heteromab | 94.62 |
| (KK on PD-1 light chain Fab region only) | |
| (PD-1 HC amino acid sequence of SEQ ID NO: 10 and | |
| LC amino acid sequence of SEQ ID NO: 17; | |
| TIGIT HC amino acid sequence of SEQ ID NO: 12 and | |
| LC amino acid sequence of SEQ ID NO: 13) | |

### Comparison of % Flow Through and % Elution of Exemplified Modified Kappa Light Chain Fab Region Formats in Kappa XL Column

Multispecific binding proteins of the present disclosure, in clarified medium or recovered from Protein A purification, may be subjected to a second purification step using a CaptureSelect™ Kappa XL (Thermo Fisher Cat. #494321001) pre-packed affinity column. Briefly, multispecific binding proteins of the present disclosure recovered from Protein A purification are subjected to a Kappa XL affinity column which has been equilibrated with a compatible buffer, such as phosphate buffered saline (PBS) at pH 7.4. The column is then washed to remove nonspecific binding components. The bound multispecific binding protein is eluted, for example, by pH gradient (such as 20 mM Tris buffer pH 7.0 to 10 mM sodium citrate buffer pH 3.0). Binding protein fractions are detected, such as by UV absorbance or SDS-PAGE, and then are pooled.

Percent flow through (% F T) and percent elution of exemplified heteromabs of the present disclosure following kappa XL column purification essentially as described herein is assessed. Briefly, various antibody formats (as set forth in Table 3) are subjected to Kappa XL column. Flow through material is considered to comprise of impurities, such as homodimers, or misassembled antibodies, whereas the Kappa XL ligand bound material (elution material) is considered to be the correctly assembled bispecific antibody.

Table 3 demonstrates the cMet parental mAb with DKK format for both light chain Fab regions abolishes binding to the Kappa XL column, thus 100% of the antibody was collected in the flow through and preventing differentiation of the desired and undesired antibody species. Likewise, the flow through % of the cMet parental mAb and cMet-BAH10 IgG1 heteromab was 2.8% and 5.5% respectively, with the majority of the antibody being bound to the Kappa XL column, and thus not allowing for differentiation between the desired and undesired antibody species. In contrast, the results in Table 3 demonstrate cMet-BAH10 IgG1 heteromab with DKK format for the cMet light chain Fab region only resulted in 29.3% flow through species and 70.7% eluted multispecific binding protein species, indicating that the DKK format for kappa light chain Fab regions allows for selective differentiation and enables separation of the desired multispecific binding protein species from the undesired species.

Furthermore, Table 3 demonstrates the DKK and the ADK kappa light chain Fab region formats on the PD-1 light chain Fab region (of the PD-1-Tigit IgG1 heteromab), results in 80.72% and 78.24% elution species respectively, thus indicating that the both formats selectively differentiate the desired elution species of multispecific binding protein from undesired (flow through) species.

TABLE 3
Kappa XL % Flow Through and % Elution comparison

| | % Flow | |
|---|---|---|
| Format | Through (FT) | % Elution |
| cMet parental mAb | 2.8 | 97.20 |
| cMet-BAH10 IgG1 heteromab | 5.5 | 94.5 |
| cMet parental mAb | 100 | No detection |
| (with DKK LC Fab region formats | | |
| for both arms) | | |
| cMet-BAH10 IgG1 heteromab (with | 29.3 | 70.7 |
| DKK LC Fab region format on cMet | | |
| arm only) | | |
| PD-1-Tigit IgG1 heteromab | 0.44 | 99.56 |
| PD-1-Tigit IgG1 heteromab (with | 19.28 | 80.72 |
| DKK LC Fab region on PD-1 arm | | |
| only) | | |
| PD-1-Tigit IgG1 heteromab | 21.76 | 78.24 |
| (with ADK LC Fab region on PD-1 | | |
| arm only) | | |
| PD-1-Tigit IgG1 heteromab (with | 26.55 | 73.45 |
| DK LC Fab region on PD-1 arm | | |
| only) | | |
| PD-1-Tigit IgG1 heteromab (with | 25.21 | 74.79 |
| KK LC Fab region on PD-1 arm | | |
| only) | | |

Together, the results demonstrate that the kappa light chain Fab region formats of Table 1a, when expressed on only one light chain Fab region of an IgG heteromab, effectively differentiates the desired multispecific binding protein from the undesired species and thus enables for effective separation and purification of the desired binding protein.

### Purified Multispecific Binding Protein Binding to Protein A and KappaXL Ligands

Protein a Binding Analysis

Binding of the heavy chain Fc region formats of Table 1b for exemplified IgG heteromabs to Protein A ligand may be assessed via ELISA. Briefly, 96-well flat bottom Elisa plates are coated with 2 ug/mL goat anti-human-kappa protein at 100 ul/well and incubated overnight at 4° C. The following day, plates are washed 3× with wash buffer 0.05% PBS-Tween 20 (PBS-T)) and blocked for 1 hr with blocking buffer (casein, 200 L/well) at room temperature (RT). Plates are washed 3× with wash buffer, and binding proteins (as shown in Table 4) are added to individual wells at 10 μg/mL and serially diluted 1:3, at a volume of 100 uL/well in PBS-T. Plates are incubated at RT for 1 hr, and washed 3× with wash buffer. Biotin-Protein A at 0.5 ug/ml is added at 100 uL/well and plates are incubated for 1 hr at RT, washed 3×, and 100 uL/well of streptavidin labeled alkaline phosphatase (SA-AP) is added to each well. Plates are incubated 30 min at RT. Plates are then washed 3×, and 100 uL/well of p-Nitrophenyl Phosphate, Disodium Salt (PNAP)(Thermo Fisher Scientific) substrate is added. Reactions are stopped and optical density is measured using a colorimetric microplate reader set to 405 nm. Results are provided in Table 4.

TABLE 4
Protein A Binding of Purified Binding Proteins

| | Binding to Protein A |
|---|---|
| Format | EC50 (nM) |
| cMet-BAH10 IgG1 heteromab | 0.48 |
| cMet-BAH10 IgG1 heteromab | 0.56 |
| (with RE HC Fc format on | |
| cMet arm only) | |
| cMet parental mAb | 0.98 |
| (with RE HC Fc format on both HCs) | |

The results demonstrate the heavy chain Fc region RE format, when expressed as part of only the cMet heavy chain Fc region of the cMet-BAH10 IgG1 heteromab, demonstrates an approx. 1.2-fold decrease in binding to the Protein A as compared to the cMet-BAH10 IgG1 heteromab. When the heavy chain Fc region RE format is expressed as part of both heavy chain Fc regions, there is an approx. 2-fold decrease of binding affinity to Protein A as compared to parental. This data demonstrates the heavy chain Fe region RE format enables elution of desired binding molecules at a higher pH and differentiation from undesired or contaminating species through differential Protein A elution.

Kappa XL Binding Analysis

Binding of the light chain Fab region formats of Table 1a for exemplified IgG hetermabs to Kappa XL ligand may be assessed via ELISA. Briefly, 96-well flat bottom ELISA plates are coated with 2 ug/mL goat anti-human-IgG protein at 100 ul/well and incubated overnight at 4° C. The following day, plates are washed 3× with wash buffer (0.05% PBS-Tween 20) and blocked for 1 hr with blocking buffer (casein, 200 μL/well) at room temperature (RT). Plates are washed 3× with wash buffer, and binding proteins (as shown in Table 5) are added at 10 μg/mL and serially diluted at 1:3 at 100 uL/well in DPBS (Dulbecco's HyClone). Plates are incubated at RT for 1 hr, are washed 3× with wash buffer and Biotin-KappaXL is added at 1 ug/ml at 100 uL/well. Plates are then incubated for 1 hr at RT, washed 3×, and 100 uL/well of SA-AP is added to each well and incubated 30 min at RT. Plates are then washed 3×, and 100 uL/well PNAP substrate is added. Reactions are stopped and the optical density is measured using a colorimetric microplate reader set to 405 nm. Results are provided in Table 5.

TABLE 5
Kappa XL Ligand Binding to Purified Binding Proteins

| | Binding to KappaXL |
|---|---|
| Format | ligand EC50 (nM) |
| cMet-BAH10 IgG1 heteromab | 0.57 |
| cMet -BAH10 IgG1 heteromab (with | 2.50 |
| DKK LC Fab region format on cMet arm only) | |
| cMet -BAH10 IgG1 heteromab (with ADK LC | 1.27 |
| Fab region format on cMet arm only) | |
| cMet parental mAb | no detectable binding |
| (with DKK LC Fab region formal on both LCs) | |

The results in Table 5 demonstrate both cMet-BAH10 IgG heteromab having DKK format as part of the cMet light chain Fab region only (2.50 nm) and cMet-BAH10 IgG heteromab having ADK as part of the cMet light chain Fab region only (1.27 nM) display lower binding affinity to Kappa XL ligand when compared to cMet-BAH10 IgG1 heteromab without the light chain Fab region formats of Table 1a (0.57 nM). The cMet parental mAb with DKK format expressed as part of both LCs had no detectable binding to Kappa XL ligand. These results demonstrated that both the DKK and ADK light chain Fab region formats, when incorporated on a single “arm” of the multispecific binding protein, decrease binding affinity to Kappa XL ligand allowing for removal of undesired or contaminating species (e.g., in flow-through). Note, this benefit may be enhanced further for multispecific binding proteins in which light chain Fab region formats of Table 1a are designed to be included only with the higher expressing “arm” of the binding protein).

### Purity, Identity and Heterogeneity Analysis of Purified Antibodies

Multispecific binding proteins comprising heavy chain Fc region and/or light chain Fab region formats of Table 1 are subjected to Protein A (step 1) purification followed by Kappa XL (step 2) purification. Flow through and elution material is analyzed for purity, identity and heterogeneity by standard techniques such as size exclusion chromatography (SEC), capillary electrophoresis (lab chip NR ceSDS), high performance liquid chromatography (HIC-HPLC) and intact mass spectrometry. SEC is used to analyze samples for percent high molecular weight (MW), percent Front shoulder, percent Main Peak, and percent low molecular weight (LMW). Percentages are calculated via Empower analysis of chromatographs using the ratio of AUC of the peaks eluted before the monomer peak to total AUC. The NR ceSDS is used to quantify levels of total Ab (%) and 12 Ab (%) in the purified material. Formats of binding proteins assessed at each step are provided in Tables 6, 7 and 8.

TABLE 6
(Step 1) Protein A Capture Material Profile

| | SEC-HPLC | | | | NR ceSDS | |
|---|---|---|---|---|---|---|
| | HMW | Front | Main | LMW | Ab | 1/2Ab |
| Format | (%) | (%) | (%) | (%) | (%) | (%) |
| PD-1-TIGIT IgG1 heteromab | 7.0 | 4.0 | 75.3 | 13.8 | 80.2 | 17.8 |
| PD-1-TIGIT IgG1 heteromab | 4.7 | 3.2 | 79.4 | 12.7 | 83.3 | 13.3 |
| (with DKK LC Fab region format | | | | | | |
| of PD-1 arm | | | | | | |
| PD-1-TIGIT IgG1 heteromab | 4.6 | 3.7 | 76.9 | 14.9 | 80.8 | 16.3 |
| (with ADK LC Fab region format | | | | | | |
| of PD-1 arm) | | | | | | |
| PD-1-TIGIT IgG1 heteromab | 3.4 | 5.3 | 73.5 | 17.8 | 78.9 | 18.4 |
| (with DK LC Fab region format | | | | | | |
| of PD-1 arm) | | | | | | |
| PD-1-TIGIT IgG1 heteromab | 3.7 | 4.2 | 76.0 | 16.1 | 78.0 | 16.3 |
| (with KK LC Fab region format | | | | | | |
| of PD-1 arm) | | | | | | |

As shown in Table 6, Step 1 (Protein A purification) shows the kappa light chain Fab region formats of Table 1a have no negative impact on the HMW species, when compared to the control (PD-1-TIGIT IgG1 heteromab), demonstrating the light chain Fab region formats of Table 1a do not negatively impact assembly of the multispecific binding protein. Furthermore, the main peak of the light chain Fab region formats of Table 1a was comparable to the control (PD-1-TIGIT IgG1 heteromab) and no significant differences were observed between the light chain Fab region double and triple formats of Table 1a.

TABLE 7
(Step 2) Kappa XL Elution Profile

| | | | | | Non reduced | | HIC- |
|---|---|---|---|---|---|---|---|
| | SEC-HPLC | | | | ceSDS | | HPLC |
| | HMW | Front | Main | LMW | Ab | 1/2Ab | Main |
| Antibody Format | (%) | (%) | (%) | (%) | (%) | (%) | (%) |
| PD-1-TIGIT IgG1 | 5.9 | 3.3 | 83.1 | 7.7 | 80.2 | 17.8 | ND |
| heteromab | | | | | | | |
| PD-1-TIGIT IgG1 | 3.6 | 0.0 | 95.7 | 0.7 | 94.3 | 4.1 | 96.1 |
| heteromab | | | | | | | |
| (DKK LC Fab region | | | | | | | |
| format on PD-1 arm | | | | | | | |
| only) | | | | | | | |
| PD-1-TIGIT IgG1 | 2.5 | 0.0 | 95.7 | 0.9 | 95.7 | 3.0 | 97.1 |
| heteromab | | | | | | | |
| (ADK LC Fab region | | | | | | | |
| format on PD-1 arm | | | | | | | |
| only) | | | | | | | |
| PD-1-TIGIT IgG1 | 2.3 | 0.0 | 97.1 | 0.7 | 95.6 | 2.6 | 96.6 |
| heteromab | | | | | | | |
| (DK LC Fab region | | | | | | | |
| format on PD-1 arm | | | | | | | |
| only) | | | | | | | |
| PD-1-TIGIT IgG1 | 2.8 | 0.0 | 96.2 | 0.9 | 94.0 | 3.8 | 95.1 |
| heteromab | | | | | | | |
| (with KK LC Fab region | | | | | | | |
| format on PD-1 arm | | | | | | | |
| only) | | | | | | | |

As shown in Table 7, Step 2 (Kappa XL purification) elution profile shows that the purity of the main peak increased to over 95% for all kappa light chain Fab region formats of Table 1a, when compared to about 83% for respective binding proteins lacking kappa light chain Fab region formats of Table 1a disclosed herein. The results further show the impurities, as demonstrated by the LMW peak, decrease to less than 1.0% for the kappa light chain Fab region formats of Table 1a compared to 7.7% for the respective binding proteins lacking kappa light chain Fab region formats of Table 1a. Similarly, the NR ceSDS profile shows the amount of full binding proteins comprising the kappa light chain Fab region formats of Table 1a was over 94% whereas binding proteins lacking kappa light chain Fab region formats of Table 1a was at only 80.2%. In addition, the ½ Ab profile for binding proteins comprising kappa light chain Fab region formats of Table 1a was less than 4% whereas binding proteins lacking kappa light chain Fab region formats of Table 1a was at 17.8%. No significant differences in elution profiles between the different binding proteins comprising the various kappa light chain Fab region formats of Table 1a was observed.

TABLE 8
(Step 2) Kappa XL Flow-Through Profile

| | SEC-HPLC | | | NR ceSDS | |
|---|---|---|---|---|---|
| | Front | Main | LMW | Ab | ½ Ab |
| Format | (%) | (%) | (%) | (%) | (%) |
| PD-1-TIGIT IgG1 heteromab | NA | NA | NA | NA | NA |
| PD-1-TIGIT IgG1 heteromab | 21.5 | 8.9 | 69.6 | 23.1 | 62.5 |
| (DKK LC Fab region format on | | | | | |
| PD-1 arm only) | | | | | |
| PD-1-TIGIT IgG1 heteromab | 22.3 | 8.8 | 68.9 | 25.0 | 51.7 |
| (ADK LC Fab region format on | | | | | |
| PD-1 arm only) | | | | | |
| PD-1-TIGIT IgG1 heteromab | 23.8 | 14.1 | 62.0 | 25.4 | 62.6 |
| (DK LC Fab region format on | | | | | |
| PD-1 arm only) | | | | | |
| PD-1-TIGIT IgG1 heteromab | 23.7 | 8.4 | 68.0 | 23.3 | 72.7 |
| (with KK LC Fab region format | | | | | |
| on PD-1 arm only) | | | | | |

Table 8 demonstrates the undesired impurities (Front and LMW %) are effectively separated out in the Kappa XL flow through and desired multispecific binding protein comprising a format of Table 1 is enriched in the elution. No unmodified control was observed in the flow through profile indicating all unmodified heteromab bound the Kappa XL column.

The results provided in Tables 3-8 demonstrate the modified kappa light chain Fab region formats of Table 1a provide for robust purification, selectively differentiating and enabling separation of desired multispecific binding protein from the undesired species and/or impurities.

### Thermal Stability Assessment of Exemplified Multispecific Binding Proteins

Thermostability of exemplified multispecific binding proteins provided herein are assessed, following Protein A and Kappa XL purification, by Differential Scanning Calorimetry (DSC). Results (unfolding temps reported as Tm1) are provided in Table 9.

TABLE 9
Thermal Stability Assessment of Exemplified Binding Proteins

| Format | Tm1 (° C.) |
|---|---|
| cMet-BAH10 IgG1 heteromab | 70.9 |
| cMet parental mAb | 71 |
| cMet parental mAb | 58.2 |
| (with RE HC Fc format on both HCs) | |
| cMet parental mAb | 70.9 |
| (with DKK LC Fab region format on both LCs) | |
| cMet-BAH10 IgG1 heteromab | 66.9 |
| (with RE HC Fc format on cMet arm) | |
| cMet-BAH10 IgG1 heteromab | 70.1 |
| (with DKK LC Fab region format on cMet arm) | |
| cMet-BAH10 IgG1 heteromab | 70.5 |
| (with ADK LC Fab region format on cMet arm) | |
| PD-1-TIGIT IgG1 heteromab | 64.59 |
| PD-1-TIGIT IgG1 heteromab | 64.5 |
| (with DKK LC Fab region format on PD-1 arm) | |
| PD-1-TIGIT IgG1 heteromab | 64.35 |
| (with ADK LC Fab region format on PD-1 arm) | |
| PD-1-TIGIT IgG1 heteromab | 64.67 |
| (with DK LC Fab region format on PD-1 arm) | |
| PD-1-TIGIT IgG1 heteromab | 64.71 |
| (with KK LC Fab region format on PD-1 arm) | |

Table 9 demonstrates the heavy chain Fc region RE format (on both arms of the cMet parental antibody) affected thermal stability, however no effect was observed when the heavy chain Fc region RE format was expressed as part of only one arm of an exemplified IgG1 heteromab. Furthermore, all modified kappa light chain Fab formats demonstrated comparable thermostability relative to respective unmodified parental molecules.

### Binding Affinity Analysis of Exemplified Multispecific Binding Proteins

Binding affinity of exemplified multispecific binding proteins provided herein is assessed via ELISA. Briefly, 384-well flat bottom Elisa plates are coated with 1 ug/mL anti-human-Fc protein at 20 ul/well and incubated overnight at 4° C. The following day, plates are washed 3× with wash buffer (0.05% PBS-Tween 20) and blocked for 1 hr with blocking buffer (casein, 60 μL/well) at room temperature (RT). Plates are washed 3× with wash buffer, and binding proteins as shown in Table 10 are added at 1 μg/mL in triplicates at 20 uL/well in DPBS (Dulbecco's HyClone). Plates are incubated at RT for 1 hr, washed 3× with wash buffer, and titrated antigens are added at 20 ul/well and incubated for 60 min at RT. Plates are washed 3×, and 20 uL/well NAAP substrate is added and incubated for 20 min. Plates are washed 3×, and PNPP substrate is added at 20 uL/well, and reactions are stopped, and optical density is measured using a colorimetric microplate reader set to 405 nm. Results are set forth in Table 10.

TABLE 10
Binding Affinity Analysis

| | Binding Affinity (to | Binding Affinity (to |
|---|---|---|
| | human TIGIT-ECD) | human PD-1-ECD) |
| Format | EC50 (nM) | EC50 (nM) |
| PD-1-TIGIT IgG1 heteromab | 0.0681 | 0.0070 |
| PD-1-TIGIT IgG1 heteromab | 0.0108 | 0.0303 |
| (ADK LC Fab region format | | |
| on PD-1 arm only) | | |
| PD-1-TIGIT IgG1 heteromab | 0.0454 | 0.0108 |
| (DKK LC Fab region format | | |
| on PD-1 arm only) | | |
| PD-1-TIGIT IgG1 heteromab | 0.0261 | 0.0097 |
| (DK LC Fab region format | | |
| on PD-1 arm only) | | |
| PD-1-TIGIT IgG1 heteromab | 0.0182 | 0.0093 |
| (with KK LC Fab region | | |
| format on PD-1 arm only) | | |

These results provided in Table 10 demonstrate the exemplified multispecific binding proteins comprising modified kappa light chain Fab region formats of Table 1a maintain comparable, and in some cases, improved binding affinity to the target antigens (as compared to unmodified parental multispecific binding protein).

### In Silico Immunogenicity Analysis

Immunogenicity of modified heavy chain Fc regions and light chain Fab region formats of exemplified multispecific binding proteins is analyzed by in silico immunogenicity analysis via Immune Epitope Database Analysis (IEDB). Immunogenicity (IG) scores and rarity scores (frequency of amino acid use at the corresponding location against human Ig repertoire) of the antibody sequences are calculated (a lower score indicative of lower immunogenicity). Results are provided in Tables 11a and 11b.

TABLE 11a
Light Chain Fab Region Immunogenicity Analysis

| | IG Score- | Rarity Score- |
|---|---|---|
| LC Format | LC | LC |
| PD-1-TIGIT IgG1 heteromab | 1.986 | 6.253 |
| PD-1-TIGIT IgG1 heteromab | 1.977 | 6.253 |
| (DKK LC Fab region format | | |
| on PD-1 arm only) | | |
| PD-1-TIGIT IgG1 heteromab | 1.982 | 6.253 |
| (ADK LC Fab region format | | |
| on PD-1 arm only) | | |
| PD-1-TIGIT IgG1 heteromab | 1.977 | 6.253 |
| (DK LC Fab region format | | |
| on PD-1 arm only) | | |
| PD-1-TIGIT IgG1 heteromab | 1.986 | 6.253 |
| (with KK LC Fab region format | | |
| on PD-1 arm only) | | |
| cMet parental mAb | 1.726 | 16.244 |
| cMet parental mAb | 1.717 | 16.244 |
| (with DKK LC Fab region - | | |
| one LC assessment) | | |

TABLE 11b
Heavy Chain Fc Region Immunogenicity Analysis

| | IG Score- | Rarity Score- |
|---|---|---|
| HC Format | HC | HC |
| cMet parental mAb | 11.518 | 28.486 |
| cMet parental mAb | 11.518 | 28.486 |
| (with RE HC Fc region - one HC assessment) | | |

The results set forth in Tables 11a and 11b show the modified light chain Fab region and heavy chain Fc region formats of Table 1a and 1b demonstrate comparable IG and rarity scores to the respective unmodified parental molecules suggesting the modified formats do not add immunogenicity risk.

Sequences

SEQ ID NO: 1 (exemplary cMet HC showing Fab region underlined)
EVOLVESGGGLVOPGGSLRLSCAASGYTFTSYWLHWVRKAPGKGLEWVGMIDP
SNSDTRFNPEFKDRFTISADTSKNTAYLQMNSLRAEDTAVYYCATYRSYVTPLDY
WGQGTLVTVSSASTKGPSVFPLAPSSKSTSGGTAALGCLVKDYFPEPVTVSWNSG
ALTSGVATGPAVLQSSGLYSLSSVVTVPSSSLGTQTYICNVNHKPSNTKVDKKVE
PKSCDKTHTCPPCPAPEAAGGPSVFLFPPKPKDTLMISRTPEVTCVVVDVSHEDPE
VI<FNWYVDGVEVHNAI<TI<PREEQYNSTYRVVSVLTVLHQDWLNGI<EYI<CI<VS
NKALPAPIEKTISKAKGQPREPQVYTLPPSREEMTDNQVSLTCLVKGFYPSDIAVE
WESNGQPENNYKTTPPVLMSDGSFFLASKLTVDKSRWQQGNVFSCSVMHEALH
NHYTQKSLSLSPGK
SEQ ID NO: 2 (exemplary cMet LC)
RIQMTQSPSSLSASVGDRVTITCKSSQSLLYTSSQKNYLAWYQDKPGKAPKLLIY
WASTRESGVPSRFSGSGSGTDFTLTISSLQPEDFATYYCQQYYAYPWTFGQGTKV
EIKRTVAAPSVFIFPPSDEQLKSGTASVVCYLNNFYPREAKVQWKVDNALQSGNS
QESVTEQDSKDSTYSLWSTLTLSKADYEKHKVYACEVTHQGLSSPVTKSFNRGE
C
SEQ ID NO: 3 (exemplary BAH10 HC showing Fab region underlined)
OVQLVOSGAEVKKPGSSVKVSCKASGYTFTTYYLHWVRYAPGOGLEWMGWIY
PGNVHAQYNEKFKGRVTITADKSTSTAYMELSSLRSEDTAVYYCARSWEGFPYW
GRGTTVTVSSASTKGPSVFPLAPCSKSTSGGTAALGCLVKDYFPEPVTVSWNSGA
LTSGVHTFPAVLQSSGLYSLSSVVTVPSSSLGTQTYICNVNHKPSNTKVDKKVEP
DSGDKTHTCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSN
KALPAPIEKTISKAKGQPRRPRVYTLPPSREEMTKNQVSLVCLVKGFYPSDIAVE
WESNGQPENNYKTTPPVLDSDGSFFLYSVLTVDKSRWQQGNVFSCSVMHEALH
NHYTQKSLSLSPGK
SEQ ID NO: 4 (exemplary BAH10 LC)
DIQMTQSPSSLSASVGDRVTITCKASQNVGINVAWYQRKPGDAPKSLISSASYRY
SGVPSRFSGSGSGTDFTLTISSLQPEDFATYFCQQYDTYPFTFGQGTKVEIKRTVA
APSVFIFPPSKEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 5 (exemplary cMet HC having RE format shown underlined and
italicized, showing Fab region underlined)
EVQLVESGGGLVQPGGSLRLSCAASGYTFTSYWLHWVRKAPGKGLEWVGMIDP
SNSDTRFNPEFKDRFTISADTSKNTAYLQMNSLRAEDTAVYYCATYRSYVTPLDY
WGOGTLVTVSSASTKGPSVFPLAPSSKSTSGGTAALGCLVKDYFPEPVTVSWNSG
ALTSGVATGPAVLQSSGLYSLSSVVTVPSSSLGTQTYICNVNHKPSNTKVDKKVE
PKSCDKTHTCPPCPAPEAAGGPSVFLFPPKPKDTLMISRTPEVTCVVVDVSHEDPE
VKFNWYVDGVEVHNAKTKPREEQYNSTYRVVSVLTVLH [symbol] DWLNG [symbol] EYKCKVS
NKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALH
NHYTQKSLSLSPGK
SEQ ID NO: 6 (exemplary cMet HC, showing Fab region underlined)
EVQLVESGGGLVQPGGSLRLSCAASGYTFTSYWLHWVRKAPGKGLEWVGMIDP
SNSDTRFNPEFKDRFTISADTSKNTAYLQMNSLRAEDTAVYYCATYRSYVTPLDY
WGQGTLVTVSSASTKGPSVFPLAPSSKSTSGGTAALGCLVKDYFPEPVTVSWNSG
ALTSGVATGPAVLQSSGLYSLSSVVTVPSSSLGTQTYICNVNHKPSNTKVDKKVE
PKSCDKTHTCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALH
NHYTQKSLSLSPGK
SEQ ID NO: 7(exemplary cMet LC having DKK format shown underlined)
RIQMTQSPSSLSASVGDRVTITCKSSQSLLYTSSQKNYLAWYQDKPGKAPKLLIY
WASTRESGVPSRFSGSGSGTDFTLTISSLQPEDFATYYCQQYYAYPWTFGQGTKV
EIKRTDAAPSVFIFPPSDEQLKSGTASVVCYLNNFYPRKAKVQWKVDNALQSGNS
QESVTEQDSKDSTYSLWSTLTLSKADYEKHKVYACEVTHKGLSSPVTKSFNRGE
C
SEQ ID NO: 8 (exemplary cMet HC having RE format shown underlined and
italicized; Fab region shown underlined)
EVQLVESGGGLVQPGGSLRLSCAASGYTFTSYWLHWVRKAPGKGLEWVGMIDP
SNSDTRFNPEFKDRFTISADTSKNTAYLQMNSLRAEDTAVYYCATYRSYVTPLDY
WGOGTLVTVSSASTKGPSVFPLAPSSKSTSGGTAALGCLVKDYFPEPVTVSWNSG
ALTSGVATGPAVLQSSGLYSLSSVVTVPSSSLGTQTYICNVNHKPSNTKVDKKVE
PKSCDKTHTCPPCPAPEAAGGPSVFLFPPKPKDTLMISRTPEVTCVVVDVSHEDPE
VKFNWYVDGVEVHNAKTKPREEQYNSTYRVVSVLTVLH [symbol] DWLNG [symbol] EYKCKVS
NKALPAPIEKTISKAKGQPREPQVYTLPPSREEMTDNQVSLTCLVKGFYPSDIAVE
WESNGQPENNYKTTPPVLMSDGSFFLASKLTVDKSRWQQGNVFSCSVMHEALH
NHYTQKSLSLSPGK
SEQ ID NO: 9 (exemplary cMet LC having ADK format shown underlined)
RIQMTQSPSSLSASVGDRVTITCSVSSSVSSIYLHWYQDKPGKAPKLLIYSTSNLAS
GVPSRFSGSGSGTDFTLTISSLQPEDFATYYCQVYSGYPLTFGGGTKVEIKRADAA
PSVFIFPPSDEQLKSGTASVVCYLNNFYPREAKVQWKVDNALQSGNSQESVTEQD
SKDSTYSLWSTLTLSKADYEKHKVYACEVTHKGLSSPVTKSFNRGEC
SEQ ID NO: 10 (exemplary PD1 HC showing Fab region underlined)
QVQLVQSGAEVKKPGSSVKVSCKASGGTFSSYAISWVRYAPGQGLEWMGLIIPSF
DTAGYAQKFQGRVAITVDESTSTAYMELSSLRSEDTAVYYCARAEHSSTGTFDY
WGRGTLVTVSSASTKGPSVFPLAPSSKSTSGGTAALGCLVADYFPEPVTVSWNSG
ALTSGVHTFPAVLQSSGLYSLASVVTVPSSSLGTQTYICNVNHKPSNTKVDERVE
PKSCDKTHTCPPCPAPEAAGGPSVFLFPPKPKDTLMISRTPEVTCVVVDVSHEDPE
VI<FNWYVDGVEVHNAKTKPREEQYNSTYRVVSVLTVLHQDWLNGKEYKCKVS
NKALAAPIEKTISKAKGQPREPQVYTLPPSREEMTDNQVSLTCLVKGFYPSDIAVE
WESNGQPENNYKTTPPVLMSDGSFFLASKLTVDKSRWQQGNVFSCSVMHEALH
NHYTQKSLSLSPGK
SEQ ID NO: 11 (exemplary PDI LC)
DIQMTQSPSSVSASVGDRVTITCRASQGISSWLAWYQRKPGDAPKLLISAASSLQS
GVPSRFSGSGSGTDFTLTISSLQPEDFATYYCQQANHLPFTFGGGTKVEIKRTVAA
PSVFIFPPSDKQLKSGTARVVCLLNNFYPREAKVQWKVDNALQSGNSQESVTEQ
DSKDSTYSLISTLTLSKADYEKHKVYACEVTHQGLSSPVTKSFNRGEC
SEQ ID NO: 12 (exemplary TIGIT HC showing Fab region underlined)
EVQLVESGGGLVQPGGSLRLSCAASGFDFSSYGVPWVRKAPGKGLEWVGYIDPI
FGPTYYADEVKGRFTISADDSKNSLYLQMNSLKTEDTAVYYCARDYSYGYAYA
LDIWGQGTLVTVSSASTKGPSVFPLAPSSKSTSGGTAALGCLVKDYFPEPVTVSW
NSGALTSGVHTFPAVLQSSGLYSLSSWTVPSSSLGTQTYICNVNHKPSNTKVDK
RVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKC
KVSNKALAAPIEKTISKAKGQPRRPRVYTLPPSREEMTKNQVSLVCLVKGFYPSDI
AVEWESNGQPENNYKTTPPVLDSDGSFFLYSVLTVDKSRWQQGNVFSCSVMHE
ALHNHYTQKSLSLSPGK
SEQ ID NO: 13 (exemplary TIGIT LC)
RIVMTQTPLSLSVTPGQPASISCQASQRISPYLAWYLDKPGQPPQLLISRASKLASG
VPDRFSGSGSGTDFTLKISRVEAEDVGVYYCQSYYVHTSSGYAFGGGTKVEIKRT
VAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
SEQ ID NO: 14 (exemplary DKK LC having DKK format shown underlined)
DIQMTQSPSSVSASVGDRVTITCRASQGISSWLAWYQRKPGDAPKLLISAASSLQS
GVPSRFSGSGSGTDFTLTISSLQPEDFATYYCQQANHLPFTFGGGTKVEIKRTDAA
PSVFIFPPSDKQLKSGTARVVCLLNNFYPRKAKVQWKVDNALQSGNSQESVTEQ
DSKDSTYSLISTLTLSKADYEKHKVYACEVTHKGLSSPVTKSFNRGEC
SEQ ID NO: 15 (exemplary PDI LC having ADK format shown underlined)
DIQMTQSPSSVSASVGDRVTITCRASQGISSWLAWYQRKPGDAPKLLISAASSLQS
GVPSRFSGSGSGTDFTLTISSLQPEDFATYYCQQANHLPFTFGGGTKVEIKRADAA
PSVFIFPPSDKQLKSGTARVVCLLNNFYPREAKVQWKVDNALQSGNSQESVTEQ
DSKDSTYSLISTLTLSKADYEKHKVYACEVTHKGLSSPVTKSFNRGEC
SEQ ID NO: 16 (exemplary PD1 LC having DK format shown underlined)
DIQMTQSPSSVSASVGDRVTITCRASQGISSWLAWYQRKPGDAPKLLISAASSLQS
GVPSRFSGSGSGTDFTLTISSLQPEDFATYYCQQANHLPFTFGGGTKVEIKRTDAA
PSVFIFPPSDKQLKSGTARVVCLLNNFYPRKAKVQWKVDNALQSGNSQESVTEQ
DSKDSTYSLISTLTLSKADYEKHKVYACEVTHQGLSSPVTKSFNRGEC
SEQ ID NO: 17 (exemplary PD1 LC having KK format shown underlined)
DIQMTQSPSSVSASVGDRVTITCRASQGISSWLAWYQRKPGDAPKLLISAASSLQS
GVPSRFSGSGSGTDFTLTISSLQPEDFATYYCQQANHLPFTFGGGTKVEIKRTVAA
PSVFIFPPSDKQLKSGTARVVCLLNNFYPRKAKVQWKVDNALQSGNSQESVTEQ
DSKDSTYSLISTLTLSKADYEKHKVYACEVTHKGLSSPVTKSFNRGEC

1. A multispecific binding protein that binds a first antigen and a second antigen, the multispecific binding protein comprising:
a first antigen binding domain comprising a first light chain Fab region and a first heavy chain Fab region, wherein the first light chain Fab region is a kappa light chain comprising:
a lysine at amino acid residue 143 (EU numbering) and a lysine at amino acid residue 199 (EU numbering);
a lysine at amino acid residue 143 (EU numbering), a lysine at amino acid residue 199 (EU numbering), and an alanine at amino acid residue 109 (EU numbering);
a lysine at amino acid residue 143 (EU numbering), a lysine at amino acid residue 199 (EU numbering), and an aspartic acid at amino acid residue 110 (EU numbering);
a lysine at amino acid residue 143 (EU numbering), a lysine at amino acid residue 199 (EU numbering), an alanine at amino acid residue 109 (EU numbering), and an aspartic acid at amino acid residue 110 (EU numbering);
an aspartic acid at amino acid residue 110 (EU numbering) and a lysine at amino acid residue 143 (EU numbering);
an aspartic acid at amino acid residue 110 (EU numbering), a lysine at amino acid residue 143 (EU numbering) and an alanine at amino acid residue 109 (EU numbering);
an aspartic acid at amino acid residue 110 (EU numbering) and a lysine at amino acid residue 199 (EU numbering);
an aspartic acid at amino acid residue 110 (EU numbering), a lysine at amino acid residue 199 (EU numbering) and an alanine at amino acid residue 109 (EU numbering);
an alanine at amino acid residue 109 (EU numbering) and a lysine at amino acid residue 143 (EU numbering);
an alanine at amino acid residue 109 (EU numbering) and a lysine at amino acid residue 199 (EU numbering); or
an alanine at amino acid residue 109 (EU numbering) and an aspartic acid at amino acid residue 110 (EU numbering); and
a second antigen binding domain comprising a second light chain Fab region and a second heavy chain Fab region,
wherein the first antigen binding domain binds the first antigen and the second antigen binding domain binds the second antigen.

2. The multispecific binding protein of claim 1, wherein:
if the first light chain Fab region comprises a lysine at amino acid residue 143 (EU numbering) and a lysine at amino acid residue 199 (EU numbering), then the second light chain Fab region does not comprise a lysine at amino acid residue 143 (EU numbering) and a lysine at amino acid residue 199 (EU numbering);
if the first light chain Fab region comprises a lysine at amino acid residue 143 (EU numbering), a lysine at amino acid residue 199 (EU numbering), and an alanine at amino acid residue 109 (EU numbering), then the second light chain Fab region does not comprise a lysine at amino acid residue 143 (EU numbering), a lysine at amino acid residue 199 (EU numbering), and an alanine at amino acid residue 109 (EU numbering);
if the first light chain Fab region comprises a lysine at amino acid residue 143 (EU numbering), a lysine at amino acid residue 199 (EU numbering), and an aspartic acid at amino acid residue 110 (EU numbering), then the second light chain Fab region does not comprise a lysine at amino acid residue 143 (EU numbering), a lysine at amino acid residue 199 (EU numbering), and an aspartic acid at amino acid residue 110 (EU numbering);
if the first light chain Fab region comprises a lysine at amino acid residue 143 (EU numbering), a lysine at amino acid residue 199 (EU numbering), an alanine at amino acid residue 109 (EU numbering), and an aspartic acid at amino acid residue 110 (EU numbering), then the second light chain Fab region does not comprise a lysine at amino acid residue 143 (EU numbering), a lysine at amino acid residue 199 (EU numbering), an alanine at amino acid residue 109 (EU numbering);
if the first light chain Fab region comprises an aspartic acid at amino acid residue 110 (EU numbering) and a lysine at amino acid residue 143 (EU numbering), then the second light chain Fab region does not comprise an aspartic acid at amino acid residue 110 (EU numbering) and a lysine at amino acid residue 143 (EU numbering);
if the first light chain Fab region comprises an aspartic acid at amino acid residue 110 (EU numbering), a lysine at amino acid residue 143 (EU numbering) and an alanine at amino acid residue 109 (EU numbering), then the second light chain Fab region does not comprise an aspartic acid at amino acid residue 110 (EU numbering), a lysine at amino acid residue 143 (EU numbering) and an alanine at amino acid residue 109 (EU numbering);
if the first light chain Fab region comprises an aspartic acid at amino acid residue 110 (EU numbering) and a lysine at amino acid residue 199 (EU numbering), then the second light chain Fab region does not comprise an aspartic acid at amino acid residue 110 (EU numbering) and a lysine at amino acid residue 199 (EU numbering);
if the first light chain Fab region comprises an aspartic acid at amino acid residue 110 (EU numbering), a lysine at amino acid residue 199 (EU numbering) and an alanine at amino acid residue 109 (EU numbering), then the second light chain Fab region does not comprise an aspartic acid at amino acid residue 110 (EU numbering), a lysine at amino acid residue 199 (EU numbering) and an alanine at amino acid residue 109 (EU numbering);
if the first light chain Fab region comprises an alanine at amino acid residue 109 (EU numbering) and a lysine at amino acid residue 143 (EU numbering), then the second light chain Fab region does not comprise an alanine at amino acid residue 109 (EU numbering) and a lysine at amino acid residue 143 (EU numbering);
if the first light chain Fab region comprises an alanine at amino acid residue 109 (EU numbering) and a lysine at amino acid residue 199 (EU numbering), then the second light chain Fab region does not comprise an alanine at amino acid residue 109 (EU numbering) and a lysine at amino acid residue 199 (EU numbering); and
if the first light chain Fab region comprises an alanine at amino acid residue 109 (EU numbering) and an aspartic acid at amino acid residue 110 (EU numbering), then the second light chain Fab region does not comprise an alanine at amino acid residue 109 (EU numbering) and an aspartic acid at amino acid residue 110 (EU numbering).

3. The multispecific binding protein of claim 1,
wherein the first light chain Fab region comprises a lysine at amino acid residue 143 (EU numbering) and a lysine at amino acid residue 199 (EU numbering).

4. The multispecific binding protein of claim 3, wherein the first light chain Fab region further comprises an aspartic acid at amino acid residue 110 (EU numbering).

5. The multispecific binding protein of claim 3, wherein the first light chain Fab region further comprises an alanine at amino acid residue 109 (EU numbering).

6. The multispecific binding protein of claim 1,
wherein the first light chain Fab region comprises an aspartic acid at amino acid residue 110 (EU numbering) and a lysine at amino acid residue 143 (EU numbering.

7. The multispecific binding protein of claim 6, wherein the first light chain Fab region further comprises an alanine at amino acid residue 109 (EU numbering).

8. The multispecific binding protein of claim 1,
wherein the first light chain Fab region comprises an aspartic acid at amino acid residue 110 (EU numbering) and a lysine at amino acid residue 199 (EU numbering.

9. The multispecific binding protein of claim 8, wherein the first light chain Fab region further comprises an alanine at amino acid residue 109 (EU numbering).

10. The multispecific binding protein of claim 1,
wherein the first light chain Fab region comprises an alanine at amino acid residue 109 (EU numbering) and a lysine at amino acid residue 143 (EU numbering).

11. The multispecific binding protein of claim 1,
wherein the first light chain Fab region comprises an alanine at amino acid residue 109 (EU numbering) and a lysine at amino acid residue 199 (EU numbering).

12. The multispecific binding protein of claim 1,
wherein the first light chain Fab region comprises an alanine at amino acid residue 109 (EU numbering) and an aspartic acid at amino acid residue 110 (EU numbering).

13. The multispecific binding protein of claim 1, wherein the first antigen binding domain further comprises a first heavy chain Fc region.

14. The multispecific binding protein of claim 1, wherein the first heavy chain Fc region comprises a human IgG1, a human IgG2 or a human IgG4 constant region.

15. The multispecific binding protein of claim 13, wherein the second antigen binding domain further comprises a second heavy chain Fc region.

16. The multispecific binding protein of claim 15, wherein the second heavy chain Fc region comprises a human IgG1, a human IgG2 or a human IgG4 constant region.

17. The multispecific binding protein of claim 13, wherein the first heavy chain Fc region comprises an arginine at amino acid residue 311 (EU numbering) and a glutamic acid at amino acid residue 317 (EU numbering).

18. The multispecific binding protein of claim 15, wherein the second heavy chain Fc region comprises an arginine at amino acid residue 311 (EU numbering) and a glutamic acid at amino acid residue 317 (EU numbering).

19. The multispecific binding protein of claim 15, wherein both the first and second heavy chain Fc regions comprise a human IgG1 constant region; both comprise a human IgG2 constant region; or both comprise a human IgG4 constant region.

20. The multispecific binding protein of claim 15, wherein both the first and second heavy chain Fc regions comprise an arginine at amino acid residue 311 (EU numbering) and a glutamic acid at amino acid residue 317 (EU numbering).

21. The multispecific binding protein of claim 1, wherein the second light chain Fab region does not comprise an alanine at amino acid residue 109; does not comprise an aspartic acid at amino acid residue 110; does not comprise a lysine at amino acid residue 143; or does not comprise a lysine at amino acid residue 199.

22. The multispecific binding protein of claim 1, wherein the second light chain Fab region is a Kappa light chain.

23. The multispecific binding protein of claim 1, wherein the second light chain Fab region is a Lambda light chain.

24. A method of purifying a multispecific binding protein comprising a first antigen binding domain comprising a light chain Fab region that binds a first antigen and a second antigen binding domain comprising a light chain Fab region that binds a second antigen
wherein the first light chain Fab region is a kappa light chain comprising:
a lysine at amino acid residue 143 (EU numbering) and a lysine at amino acid residue 199 (EU numbering);
an aspartic acid at amino acid residue 110 (EU numbering) and a lysine at amino acid residue 143 (EU numbering);
an aspartic acid at amino acid residue 110 (EU numbering) and a lysine at amino acid residue 199 (EU numbering);
an alanine at amino acid residue 109 (EU numbering) and a lysine at amino acid residue 143 (EU numbering);
an alanine at amino acid residue 109 (EU numbering) and a lysine at amino acid residue 199 (EU numbering); or
an alanine at amino acid residue 109 (EU numbering) and an aspartic acid at amino acid residue 110 (EU numbering);
the method comprising:
expressing the multispecific binding protein, wherein the first antigen binding domain assembles with the second antigen binding domain; and
subjecting the multispecific binding protein to an affinity chromatography column; and
recovering purified multispecific binding protein.

25. The method of claim 24, wherein the first light chain Fab region is a kappa light chain comprising a lysine at amino acid residue 143 (EU numbering) and a lysine at amino acid residue 199 (EU numbering), and the first antigen binding domain further comprises an alanine at amino acid residue 109 (EU numbering).

26. The method of claim 24, wherein the first light chain Fab region is a kappa light chain comprising a lysine at amino acid residue 143 (EU numbering) and a lysine at amino acid residue 199 (EU numbering), and the first antigen binding domain further comprises an alanine at amino acid residue 110 (EU numbering).

27. (canceled)

28. The method of claim 24, wherein the first light chain Fab region is a kappa light chain comprising an aspartic acid at amino acid residue 110 (EU numbering) and a lysine at amino acid residue 199 (EU numbering), and the first antigen binding domain further comprises an alanine at amino acid residue 109 (EU numbering).

29. (canceled)

30. The method of claim 24, wherein the first light chain Fab region is a kappa light chain comprising an aspartic acid at amino acid residue 110 (EU numbering) and a lysine at amino acid residue 199 (EU numbering), and the first antigen binding domain further comprises an alanine at amino acid residue 109 (EU numbering).

31.-33. (canceled)

34. The method of claim 24, wherein the first antigen binding domain comprises a first heavy chain Fc region.

35. (canceled)

36. The method of claim 34, wherein the second antigen binding domain comprises a second heavy chain Fc region.

37. (canceled)

38. The method of claim 34, wherein the first heavy chain Fc region comprises an arginine at amino acid residue 311 (EU numbering) and a glutamic acid at amino acid residue 317 (EU numbering).

39. The method of claim 34, wherein the second heavy chain Fc region comprises an arginine at amino acid residue 311 (EU numbering) and a glutamic acid at amino acid residue 317 (EU numbering).

40. (canceled)

41. The method of claim 36, wherein both the first heavy chain Fc region and the second heavy chain Fc region comprise arginine at amino acid residues 311 (EU numbering) and glutamic acid at amino acid residues 317 (EU numbering).

42. The method of claim 24, wherein the second light chain Fab region does not comprise an alanine at amino acid residue 109; does not comprise an aspartic acid at amino acid residue 110; does not comprise a lysine at amino acid residue 143; or does not comprise a lysine at amino acid residue 199.

43. The method of claim 24, wherein the second light chain Fab region does not comprise an alanine at amino acid residue 109; does not comprise an aspartic acid at amino acid residue 110; does not comprise a lysine at amino acid residue 143; and does not comprise a lysine at amino acid residue 199.

44. (canceled)

45. (canceled)

46. The method of claim 24, wherein the affinity chromatography column comprises a kappa affinity ligand.

47. The method of claim 24, wherein the affinity chromatography column comprises a lambda affinity ligand.

48. The method of claim 24, wherein the affinity chromatography column comprises Protein A.

49. The method of any one of claim 24, wherein the second light chain Fab region binds to the affinity chromatography column with greater affinity than the first light chain Fab region.

50. The method of claim 24, wherein the first light chain Fab region does not bind to the affinity chromatography column.

51. The method of claim 24, further comprising:
subjecting the purified multispecific binding protein to a second affinity chromatography column after the step of recovering purified multispecific binding protein; and
recovering purified multispecific binding protein after the step of subjecting the purified multispecific binding protein to a second affinity chromatography column.

52. The method of 51, wherein the second affinity chromatography column comprises a kappa affinity ligand.

53. The method of claim 51, wherein the second affinity chromatography column comprises a lambda affinity ligand.

54. The method of claim 51, wherein the second affinity chromatography column comprises Protein A.

55. The method of claim 51, wherein the second light chain Fab region binds to the second affinity chromatography column with greater affinity than the first light chain Fab region.

56. The method of claim 51, wherein the first light chain Fab region does not bind to the second affinity chromatography column.


SEQUENCE LISTING (17 sequences)

SEQ ID NO: 1 - protein, 449 aa, Artificial Sequence
  synthetic construct
EVQLVESGGGLVQPGGSLRLSCAASGYTFTSYWLHWVRKAPGKGLEWVGMIDPSNSDTRFNPEFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTDNQVSLTCLVKGFYPSDIAVEWESNGQPENNYKTTPPVLMSDGSFFLASKLTVDKSRWQQGNVFSCSVMHEALHNHYTQKSLSLSPGK

SEQ ID NO: 2 - protein, 220 aa, Artificial Sequence
  synthetic construct
RIQMTQSPSSLSASVGDRVTITCKSSQSLLYTSSQKNYLAWYQDKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCYLNNFYPREAKVQWKVDNALQSGNSQESVTEQDSKDSTYSLWSTLTLSKADYEKHKVYACEVTHQGLSSPVTKSFNRGEC

SEQ ID NO: 3 - protein, 446 aa, Artificial Sequence
  synthetic construct
QVQLVQSGAEVKKPGSSVKVSCKASGYTFTTYYLHWVRYAPGQGLEWMGWIYPGNVHAQYNEKFKGRVTITADKSTSTAYMELSSLRSEDTAVYYCARSWEGFPYWGRGTTVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTKNQVSLVCLVKGFYPSDIAVEWESNGQPENNYKTTPPVLDSDGSFFLYSVLTVDKSRWQQGNVFSCSVMHEALHNHYTQKSLSLSPGK

SEQ ID NO: 4 - protein, 214 aa, Artificial Sequence
  synthetic construct
DIQMTQSPSSLSASVGDRVTITCKASQNVGINVAWYQRKPGDAPKSLISSASYRYSGVPSRFSGSGSGTDFTLTISSLQPEDFATYFCQQYDTYPFTFGQGTKVEIKRTVAAPSVFIFPPSKEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 449 aa, Artificial Sequence
  synthetic construct
EVQLVESGGGLVQPGGSLRLSCAASGYTFTSYWLHWVRKAPGKGLEWVGMIDPSNSDTRFNPEFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGEEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 449 aa, Artificial Sequence
  synthetic construct
EVQLVESGGGLVQPGGSLRLSCAASGYTFTSYWLHWVRKAPGKGLEWVGMIDPSNSDTRFNPEFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 220 aa, Artificial Sequence
  synthetic construct
RIQMTQSPSSLSASVGDRVTITCKSSQSLLYTSSQKNYLAWYQDKPGKAPKLLIYWASTRESGVPSRFSGSGSGTDFTLTISSLQPEDFATYYCQQYYAYPWTFGQGTKVEIKRTDAAPSVFIFPPSDEQLKSGTASVVCYLNNFYPRKAKVQWKVDNALQSGNSQESVTEQDSKDSTYSLWSTLTLSKADYEKHKVYACEVTHKGLSSPVTKSFNRGEC

SEQ ID NO: 8 - protein, 449 aa, Artificial Sequence
  synthetic construct
EVQLVESGGGLVQPGGSLRLSCAASGYTFTSYWLHWVRKAPGKGLEWVGMIDPSNSDTRFNPEFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGEEYKCKVSNKALPAPIEKTISKAKGQPREPQVYTLPPSREEMTDNQVSLTCLVKGFYPSDIAVEWESNGQPENNYKTTPPVLMSDGSFFLASKLTVDKSRWQQGNVFSCSVMHEALHNHYTQKSLSLSPGK

SEQ ID NO: 9 - protein, 215 aa, Artificial Sequence
  synthetic construct
RIQMTQSPSSLSASVGDRVTITCSVSSSVSSIYLHWYQDKPGKAPKLLIYSTSNLASGVPSRFSGSGSGTDFTLTISSLQPEDFATYYCQVYSGYPLTFGGGTKVEIKRADAAPSVFIFPPSDEQLKSGTASVVCYLNNFYPREAKVQWKVDNALQSGNSQESVTEQDSKDSTYSLWSTLTLSKADYEKHKVYACEVTHKGLSSPVTKSFNRGEC

SEQ ID NO: 10 - protein, 450 aa, Artificial Sequence
  synthetic construct
QVQLVQSGAEVKKPGSSVKVSCKASGGTFSSYAISWVRYAPGQGLEWMGLIIPSFDTAGYAQKFQGRVAITVDESTSTAYMELSSLRSEDTAVYYCARAEHSSTGTFDYWGRGTLVTVSSASTKGPSVFPLAPSSKSTSGGTAALGCLVADYFPEPVTVSWNSGALTSGVHTFPAVLQSSGLYSLASVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTDNQVSLTCLVKGFYPSDIAVEWESNGQPENNYKTTPPVLMSDGSFFLASKLTVDKSRWQQGNVFSCSVMHEALHNHYTQKSLSLSPGK

SEQ ID NO: 11 - protein, 214 aa, Artificial Sequence
  synthetic construct
DIQMTQSPSSVSASVGDRVTITCRASQGISSWLAWYQRKPGDAPKLLISAASSLQSGVPSRFSGSGSGTDFTLTISSLQPEDFATYYCQQANHLPFTFGGGTKVEIKRTVAAPSVFIFPPSDKQLKSGTARVVCLLNNFYPREAKVQWKVDNALQSGNSQESVTEQDSKDSTYSLISTLTLSKADYEKHKVYACEVTHQGLSSPVTKSFNRGEC

SEQ ID NO: 12 - protein, 451 aa, Artificial Sequence
  synthetic construct
EVQLVESGGGLVQPGGSLRLSCAASGFDFSSYGVPWVRKAPGKGLEWVGYIDPIFGPTYYADEVKGRFTISADDSKNSLYLQMNSLKTEDTAVYYCARDYSYGYAYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPRRPRVYTLPPSREEMTKNQVSLVCLVKGFYPSDIAVEWESNGQPENNYKTTPPVLDSDGSFFLYSVLTVDKSRWQQGNVFSCSVMHEALHNHYTQKSLSLSPGK

SEQ ID NO: 13 - protein, 217 aa, Artificial Sequence
  synthetic construct
RIVMTQTPLSLSVTPGQPASISCQASQRISPYLAWYLDKPGQPPQLLISRASKLASGVPDRFSGSGSGTDFTLKISRVEAEDVGVYYCQSYYVHTSSGYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 214 aa, Artificial Sequence
  synthetic construct
DIQMTQSPSSVSASVGDRVTITCRASQGISSWLAWYQRKPGDAPKLLISAASSLQSGVPSRFSGSGSGTDFTLTISSLQPEDFATYYCQQANHLPFTFGGGTKVEIKRTDAAPSVFIFPPSDKQLKSGTARVVCLLNNFYPRKAKVQWKVDNALQSGNSQESVTEQDSKDSTYSLISTLTLSKADYEKHKVYACEVTHKGLSSPVTKSFNRGEC

SEQ ID NO: 15 - protein, 214 aa, Artificial Sequence
  synthetic construct
DIQMTQSPSSVSASVGDRVTITCRASQGISSWLAWYQRKPGDAPKLLISAASSLQSGVPSRFSGSGSGTDFTLTISSLQPEDFATYYCQQANHLPFTFGGGTKVEIKRADAAPSVFIFPPSDKQLKSGTARVVCLLNNFYPREAKVQWKVDNALQSGNSQESVTEQDSKDSTYSLISTLTLSKADYEKHKVYACEVTHKGLSSPVTKSFNRGEC

SEQ ID NO: 16 - protein, 214 aa, Artificial Sequence
  synthetic construct
DIQMTQSPSSVSASVGDRVTITCRASQGISSWLAWYQRKPGDAPKLLISAASSLQSGVPSRFSGSGSGTDFTLTISSLQPEDFATYYCQQANHLPFTFGGGTKVEIKRTDAAPSVFIFPPSDKQLKSGTARVVCLLNNFYPRKAKVQWKVDNALQSGNSQESVTEQDSKDSTYSLISTLTLSKADYEKHKVYACEVTHQGLSSPVTKSFNRGEC

SEQ ID NO: 17 - protein, 214 aa, Artificial Sequence
  synthetic construct
DIQMTQSPSSVSASVGDRVTITCRASQGISSWLAWYQRKPGDAPKLLISAASSLQSGVPSRFSGSGSGTDFTLTISSLQPEDFATYYCQQANHLPFTFGGGTKVEIKRTVAAPSVFIFPPSDKQLKSGTARVVCLLNNFYPRKAKVQWKVDNALQSGNSQESVTEQDSKDSTYSLISTLTLSKADYEKHKVYACEVTHKGLSSPVTKSFNRGEC